(19) United States
(12) Patent Application Publication
(10) Pub. No.: US 2025/0353806 A1
(43) Pub. Date: Nov. 20, 2025

(54) SYNTHESIS OF LEVULINIC ACID BY HYDRATION OF FURFURYL ALCOHOL IN THE PRESENCE OF A HOMOGENEOUS ACID CATALYST AND OF A SOLVENT BASED ON ETHER AND/OR ACETALS

(71) Applicant: IFP ENERGIES NOUVELLES, Rueil-Malmaison (FR)

(72) Inventors: Pierre-Alain BREUIL, Rueil-Malmaison Cedex (FR); Kim LARMIER, Rueil-Malmaison Cedex (FR)

(73) Assignee: IFP ENERGIES NOUVELLES, Rueil-Malmaison (FR)

(21) Appl. No.: 18/869,867

(22) PCT Filed: May 11, 2023
(86) PCT No.: PCT/EP2023/062616

§ 371 Date: Nov. 27, 2024

(30) Foreign Application Priority Data
May 31, 2022 (FR) FR2205180

(51) Int. Cl. C07C 51/44

(52) U.S. Cl. CPC C07C 51/44

(57) ABSTRACT

The present invention relates to a process for synthesizing levulinic acid by hydration of furfuryl alcohol at a temperature of between 25 and 140° C. in the presence of a homogeneous acid catalyst and of an ether- and/or acetal-based solvent. The use of such a solvent makes it possible to obtain an equivalent or even better yield compared to those obtained with known solvents, while at the same time exhibiting high stability properties.

### TECHNICAL FIELD

The present invention relates to a process for synthesizing levulinic acid by hydration of furfuryl alcohol in the presence of a homogeneous acid catalyst and of an ether- or acetal-based solvent.

### PRIOR ART

Levulinic acid (also known as 4-oxopentanoic acid or γ-ketovaleric acid) is an organic product corresponding to the formula:

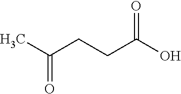

Levulinic acid is a product or a chemical intermediate that may be used in the petrochemical industry, the refining of petroleum products, the agricultural industry, the pharmaceutical industry, the food industry, the hygiene and cosmetics industry or also in the polymers and additives industry.

Levulinic acid is generally produced in two ways.

The first route, the sugar/biomass route, is the production of levulinic acid by acid hydrolysis starting from C6 or C5 sugars which may themselves be obtained from lignocellulosic biomass by acid hydrolysis, as described for example in Biofuels, Bioproducts and Biorefining 5 198-214 (2011). In addition to levulinic acid, the biomass or sugar hydrolyzates generally also contain compounds having a low boiling point, such as formic acid, acetic acid and propionic acid.

The second route is the hydration of furfuryl alcohol in the presence of a homogeneous or heterogeneous acid catalyst. This synthesis is described for example in FR2640263, U.S. Pat. No. 3,752,849 and U.S. Pat. No. 2,780,588. The use of homogeneous catalysts generally leads to higher yields of levulinic acid. In addition, heterogeneous catalysts can become fouled with humins that are formed during the synthesis, leading to a drop in acid yield.

More particularly, the patent U.S. Pat. No. 3,752,849 describes the synthesis of levulinic acid by hydration of furfuryl alcohol in the presence of an acid chosen from hydrochloric acid or oxalic acid in the presence of a solvent based on an aliphatic ketone, such as acetone, methyl ethyl ketone, diethyl ketone, methyl isobutyl ketone, or cyclohexanone. This document states that the presence of the solvent makes it possible to limit the formation of an undesirable polymer, ensuring selective production of levulinic acid with high yields. An additional organic solvent having a boiling point greater than water, especially toluene, xylene, benzene or cumene, may also be present.

The patent FR2640263 also describes the synthesis of levulinic acid by hydration of furfuryl alcohol in the presence of an acid, but in the absence of a solvent. This document states that the absence of a solvent avoids the formation of various byproducts that result from aldolization and condensation side reactions that involve said solvent. According to this document, the absence of the solvent makes it possible to increase the yield and the purity of the levulinic acid obtained.

However, the yield of such processes, whether these be via the hydration of furfuryl alcohol route or the sugar/biomass route, is in fact rather low, mainly because of the formation of numerous reaction byproducts, from which the levulinic acid must be separated by complex separation and purification processes. In addition to various low-molecular-weight byproducts, the thermal treatment at acidic pH leads to the formation of humins, which are high-molecular-weight polymeric compounds resulting from condensation reactions. Humins are generally separated in the form of solids, generally of a dark colour, which present a number of problems during the process of recovering the levulinic acid, notably via fouling of the equipment which can lead to complete clogging. Moreover, the viscosity of the humins, which increases as a function of the heating time during the synthesis step or a downstream thermal separation step, contributes to significant fouling of the separation equipment and/or to degrading the capacity for recovering the levulinic acid.

Another problem is the heat sensitivity of the levulinic acid itself, which is converted during the synthesis step or in a thermal separation step such as a downstream distillation into undesired byproducts, for example by dehydration of the levulinic acid into angelica lactone. Such conversions lower the recovery rate of the levulinic acid.

The presence of a solvent in a reaction leading to a heat-sensitive product such as levulinic acid is generally desirable since it makes it possible to limit the heating temperature and thus the degradation of the levulinic acid and/or the formation of humins. In addition, the presence of a solvent makes it possible to dissolve the byproducts, especially the humins, to a certain degree. However, the solvent itself may also be heat-sensitive or reactive under acidic conditions, and undergo degradations into byproducts that are constraining for the downstream separation and purification steps. The choice of solvent can therefore have an influence on the yield of levulinic acid.

The present invention aims to propose a process for synthesizing levulinic acid by hydration of furfuryl alcohol in the presence of a homogeneous acid catalyst and of a particular solvent, particularly an ether-and/or acetal-based solvent.

### SUMMARY OF THE INVENTION

More precisely, the invention relates to a process for synthesizing levulinic acid by hydration of furfuryl alcohol at a temperature of between 25 and 140° C. in the presence of a homogeneous acid catalyst and of an ether- and/or acetal-based solvent.

The present invention is based in particular on the act of using an ether- and/or acetal-based solvent during the synthesis of the levulinic acid by hydration of furfuryl alcohol. The use of such a solvent makes it possible to obtain an equivalent or even better yield compared to those obtained with known solvents of aliphatic ketone type, while at the same time exhibiting high stability properties. Specifically, the degradation of the solvent is avoided or limited, which facilitates recycling thereof.

Moreover, the formation of byproducts that are constraining for the downstream separation and purification steps is effectively limited.

According to a variant, the ether-and/or acetal-based solvent is chosen from the compounds corresponding to one or the other of the structures I and II, taken alone or as a mixture:

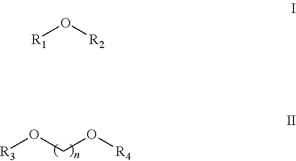

in which R₁, R₂, R₃ and R₄ are independently chosen from:
linear or branched aliphatic groups of 1 to 6 carbon atoms, optionally substituted by alkoxy groups,
cyclic or polycyclic aliphatic groups of 5 to 12 carbon atoms, optionally substituted by alkoxy or alkyl groups,
linear or branched olefinic groups of 1 to 6 carbon atoms, optionally substituted by alkoxy groups,
aromatic or polyaromatic groups of 6 to 12 carbon atoms, R₁ and R₂ may be bonded together by covalent bonds so as to form a ring, R₃ and R₄ may be bonded together by covalent bonds so as to form a ring, n is an integer between 1 and 6.

According to a variant, the solvent is chosen from diethyl ether, diisopropyl ether, diisobutyl ether, dibutyl ether, diphenyl ether, 2-methoxy-2-methylpropane, 2-methoxy-2-methylbutane, 2,5-dihydrofuran, tetrahydrofuran, 2-methyltetrahydrofuran, 3-methyltetrahydrofuran, 2,3-dihydropyran, tetrahydropyran, 1,3-dioxolane, 1,3-dioxane, 1,4-dioxane, 1,2-dimethoxyethane, benzofuran, 2,2-dimethoxypropane, 2,2-di(2-ethylhexyloxy) propane, 2-methoxytetrahydrofuran and di(2-methoxyethyl) ether, taken alone or as a mixture.

According to a variant, the homogeneous acid catalyst is chosen from a homogeneous, organic or inorganic Brønsted acid.

According to a variant, the homogeneous acid catalyst is hydrochloric acid.

According to a variant, water is present in an amount such that the water/furfuryl alcohol molar ratio is between 0.9 and 10.0 mol/mol.

According to a variant, the solvent is present in an amount such that the solvent/furfuryl alcohol molar ratio is between 0.1 and 5 mol/mol.

According to a variant, the homogeneous acid catalyst is present in an amount such that the acid/furfuryl alcohol molar ratio is between 0.01 and 1.0 mol/mol.

According to a variant, the process is carried out at a temperature of between 60 and 110° C.

According to a variant, the process is carried out at a pressure of between 0.01 MPa and 1 MPa.

According to a variant, the reaction effluent resulting from the synthesis is subjected to at least one separation step.

According to a variant, the reaction effluent resulting from the synthesis is subjected to at least one thermal separation step.

According to a variant, the reaction effluent resulting from the synthesis is subjected to at least one step of thermal separation in the presence of a flux having a boiling point greater than that of the levulinic acid.

According to a variant, the flux has a boiling range of between 250 and 620° C. and is of petroleum origin and/or of vegetable origin and/or based on polymers or a mixture thereof.

According to a variant, the flux is chosen from a petroleum cut chosen from a vacuum gas oil, a heavy oil obtained from a fluidized-bed catalytic cracking, a settling oil, an unconverted oil originating from a hydrocracker, or a polyethylene glycol having an average molar mass of greater than or equal to 600 g/mol.

In the present description, the term “comprise” is synonymous with (means the same thing as) “include” and “contain”, and is inclusive or open-ended and does not exclude other elements which are not mentioned. It is understood that the term “to comprise” includes the exclusive and closed term “to consist of”.

In the present description, the expression “of between . . . and . . . ” means that the limiting values of the interval are included in the described range of values, unless specified otherwise.

In the present invention, the different ranges of values of given parameters can be used alone or in combination. For example, a preferred range of pressure values can be combined with a more preferred range of temperature values, or a preferred range of values for one chemical compound or element can be combined with a more preferred range of values for another chemical compound or element.

Hereinafter, particular and/or preferred embodiments of the invention may be described. They can be employed separately or combined together, without limitation of combination when this is technically feasible.

In the present invention, the boiling temperature is measured under standard conditions, namely at 1 atmosphere, or 760.00 mmHg. At this pressure, the boiling temperature of pure water is 100° C. and the boiling point of levulinic acid is 245° C.

### DETAILED DESCRIPTION

### Synthesis of Levulinic Acid

The invention relates to a process for synthesizing levulinic acid by hydration of furfuryl alcohol at a temperature of between 25 and 140° C. in the presence of a homogeneous acid catalyst and of an ether- and/or acetal-based solvent according to the following formula:

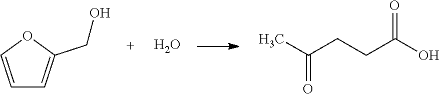

The synthesis by hydration of furfuryl alcohol can be implemented in a continuously operating or non-continuously operating unit.

When the synthesis is implemented in a continuously operating unit, the furfuryl alcohol is introduced into the reactor by pouring, by injection or by any other means, on the one hand, and the solvent, water and acid mixture is introduced by pouring, by injection or by any other means, on the other hand, taking into account a target residence time. The withdrawal of the reaction effluent containing the levulinic acid formed is carried out continuously at the same time.

The synthesis by hydration of furfuryl alcohol may also be implemented in a unit operating as a reactor that is continuously fed, over the course of which no withdrawal of the contents of the reactor is carried out, i.e. in “fed batch” mode.

In the case of a fed-batch mode, the furfuryl alcohol is introduced into the reactor continuously, by pouring, by injection or by any other means, into the unit containing the water, the acid catalyst and the solvent. The reaction medium may be stirred. At the end of the reaction, the reaction effluent containing the levulinic acid formed can be sent continuously into a separation section as described below.

The synthesis by hydration of furfuryl alcohol may also be implemented in a unit operating as a closed reactor, i.e. in “batch” mode.

In the case of a batch mode, all of the compounds (furfuryl alcohol, water, solvent, acid catalyst) are placed in a reactor, and then the reaction is carried out while heating. At the end of the reaction, the reaction effluent containing the levulinic acid formed can be sent into a separation section as described below.

In the case of continuous operation, the compound (i.e. water or acid or solvent) to furfuryl alcohol molar ratio corresponds to the molar flow rate of said compound entering the reactor in relation to the molar flow rate of furfuryl alcohol entering the reactor.

In the case of fed-batch operation, the compound (i.e. water or acid or solvent) to furfuryl alcohol molar ratio corresponds to the total amount of said compound introduced into the reactor during the whole of the reaction in relation to the total amount of furfuryl alcohol introduced into the reactor during the whole of the reaction.

Independently of the amounts of material used for the synthesis, the duration of addition corresponds to the duration over which the furfuryl alcohol is introduced into the reaction section. This addition can be performed continuously or batchwise. The duration of addition is generally between 5 minutes and 4 days, preferably between 1 hour and 2 days, very preferably between 2 hours and 1 day.

At the end of the reaction the reaction effluent can be stirred under the temperature and pressure conditions of the reaction for a maturation time. The maturation phase is generally between 1 second and 4 days, preferably between 1 minute and 2 days, very preferably between 5 minutes and 1 day. At the end of this maturation phase, the reaction effluent containing the levulinic acid formed can be sent into a separation section as described below.

The furfuryl alcohol may be biobased or non-biobased. It may, for example, be obtained from C5 sugars (comprising 5 carbon atoms) or C6 sugars (comprising 6 carbon atoms).

The water is usually present in an amount such that the water/furfuryl alcohol molar ratio is between 0.9 and 10.0 mol/mol, preferably between 1.0 and 5.0 mol/mol, very preferably between 1.1 and 3.0 mol/mol.

According to the invention, the process is carried out in the presence of at least one homogeneous acid catalyst. The acid catalyst is generally chosen from homogeneous, organic or inorganic Brønsted acids.

In one embodiment, at least one catalyst is chosen from homogeneous organic Brønsted acids.

Preferably, the homogeneous organic Brønsted acid catalysts are chosen from organic acids of general formulae R′COOH, R′SO₂H, R′SO₃H, (R′SO₂) NH, (R′O)₂PO₂H, R′OH, in which R′ is chosen from the following groups:
alkyls, preferably comprising between 1 and 15 carbon atoms, preferably between 1 and 10 and preferably between 1 and 6, which are or are not substituted by at least one substituent chosen from a hydroxyl, an amine, a nitro, a halogen, preferably fluorine, and an alkyl halide,
alkenyls, which are or are not substituted by at least one group chosen from a hydroxyl, an amine, a nitro, an oxo, a halogen, preferably fluorine, and an alkyl halide,.
aryls comprising between 5 and 15 carbon atoms and preferably between 6 and 12 carbon atoms, which are or are not substituted by a substituent chosen from a hydroxyl, an amine, a nitro, an oxo, a halogen, preferably fluorine, and an alkyl halide,.
heteroaryls comprising between 4 and 15 carbon atoms and preferably between 4 and 12 carbon atoms, which are or are not substituted by a substituent chosen from a hydroxyl, an acid, an amine, a nitro, an oxo, a halogen, preferably fluorine, and an alkyl halide.

When the catalysts of organic Brønsted acid type are chosen from organic acids of general formula R′—COOH, R′ can also be a hydrogen.

Preferably, the organic Brønsted acids are chosen from formic acid, acetic acid, trifluoroacetic acid, lactic acid, levulinic acid, 2,5-furandicarboxylic acid, methanesulfinic acid, methanesulfonic acid, trifluoromethanesulfonic acid, bis(trifluoromethanesulfonyl)amine, benzoic acid, para-toluenesulfonic acid, 4-biphenylsulfonic acid, diphenyl phosphate and 1, 1′-binaphthyl-2,2′-diyl hydrogen phosphate. Very preferably, the homogeneous organic Brønsted acid catalyst is chosen from methanesulfonic acid (CH₃SO₃H), para-toluenesulfonic acid and trifluoromethanesulfonic acid (CF₃SO₃H).

In one embodiment, at least one catalyst is chosen from homogeneous inorganic Brønsted acids.

Preferably, the homogeneous inorganic Brønsted catalysts are chosen from HF, HCl, HBr, HI, H₂SO₃, H₂SO₄, H₃PO₂, H₃PO₄, HNO₂, HNO₃, H₂WO₄, H₄SiW₁₂O₄₀, H₃PW₁₂O₄₀, (NH₄)₆(W₁₂O₄₀).xH₂O, H₄SiMo₁₂O₄₀, H₃PMo₁₂O₄₀, (NH₄)₆Mo₇O₂₄.xH₂O, H₂MoO₄, HReO₄, H₂CrO₄, H₂SnO₃, H₄SiO₄, H₃BO₃, HClO₄, HBF₄, HSbF₅, HPF₆, H₂FO₃P, ClSO₃H, FSO₃H, HN(SO₂F)₂ and HIO₃. Preferably, the inorganic Brønsted acids are chosen from HCl, HBr, HI, H₂SO₄, H₃PO₄ or HNO_{3.} Very preferably, the inorganic Brønsted acid is hydrochloric acid HCl.

The acid catalyst is usually present in an amount such that the acid/furfuryl alcohol molar ratio is between 0.01 and 1.0 mol/mol, preferably between 0.02 and 0.5 mol/mol.

The synthesis by hydration of furfuryl alcohol is carried out in the presence of an ether- and/or acetal-based solvent.

The solvent is preferably a solvent in which the water is partly or completely soluble. The solvent advantageously has a lower boiling point than that of levulinic acid, which makes it possible to separate the solvent for recycling and to limit the heating temperature when the optional preliminary thermal separation step is carried out, thus avoiding the formation of humins and/or the degradation of the levulinic acid.

The solvent advantageously has a boiling point of greater than 75° C., preferably of greater than 80° C. The formation of levulinic acid by hydration of furfuryl alcohol is generally favored at an elevated temperature while avoiding an excessively high temperature that leads to the formation of humins and/or to the degradation of the levulinic acid.

According to a variant, the ether- and/or acetal-based solvent is chosen from the compounds corresponding to one or the other of the structures I and II, taken alone or as a mixture:

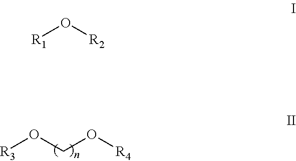

in which R₁, R₂, R₃ and R₄ are independently chosen from:
linear or branched aliphatic groups of 1 to 6 carbon atoms, optionally substituted by alkoxy groups,
cyclic or polycyclic aliphatic groups of 5 to 12 carbon atoms, optionally substituted by alkoxy or alkyl groups,
linear or branched olefinic groups of 1 to 6 carbon atoms, optionally substituted by alkoxy groups,
aromatic or polyaromatic groups of 6 to 12 carbon atoms,

R₁ and R₂ may be bonded together by covalent bonds so as to form a ring, R₃ and R₄ may be bonded together by covalent bonds so as to form a ring, n is an integer between 1 and 6.

According to a variant, the solvent is an ether-based solvent and is chosen from diethyl ether, diisopropyl ether, diisobutyl ether, dibutyl ether, diphenyl ether, 2-methoxy-2-methylpropane, 2-methoxy-2-methylbutane, 2,5-dihydrofuran, tetrahydrofuran, 2-methyltetrahydrofuran, 3-methyltetrahydrofuran, 2,3-dihydropyran, tetrahydropyran, 1,3-dioxolane, 1,3-dioxane, 1,4-dioxane, 1,2-dimethoxyethane and benzofuran, taken alone or as a mixture.

According to another variant, the solvent is an acetal-based solvent and is chosen from 2,2-dimethoxypropane, 2,2-di(2-ethylhexyloxy) propane, 2-methoxytetrahydrofuran and di(2-methoxyethyl) ether, taken alone or as a mixture.

According to another variant, the solvent is a mixture of an ether-and acetal-based solvent, chosen from one of the ethers and acetals mentioned above, in any proportion.

Preferably, the solvent is chosen from diisobutyl ether, dibutyl ether, 2-methyltetrahydrofuran, 3-methyltetrahydrofuran, 1,4-dioxane and 1,2-dimethoxyethane, taken alone or as a mixture.

Even more preferably, the solvent is chosen from 1,4-dioxane and 1,2-dimethoxyethane, taken alone or as a mixture.

The solvent is usually present in an amount such that the solvent/furfuryl alcohol molar ratio is between 0.1 and 5 mol/mol, preferably between 0.5 and 3 mol/mol, very preferably between 1 and 2 mol/mol.

The synthesis by hydration of furfuryl alcohol is generally carried out at a temperature of between 25 and 140° C., preferably of between 40 and 120° C., very preferably of between 60 and 110° C.

The synthesis by hydration of furfuryl alcohol is generally carried out at a pressure of between 0.01 MPa and 1 MPa (0.1 bara and 10 bara), and preferably at atmospheric pressure.

Preferably, the levulinic acid is synthesized by hydration of furfuryl alcohol in the presence of a homogeneous acid catalyst, preferably hydrochloric acid, and of an ether-based solvent, preferably chosen from 1,4-dioxane and/or 1,2-dimethoxyethane.

The conversion of the furfuryl alcohol is generally greater than 95%, preferably greater than 98%, very preferably greater than 99%.

The yield of levulinic acid is defined as the ratio of the obtained molar concentration of levulinic acid to the molar concentration of furfuryl alcohol used in the reaction medium, expressed in %. The yield of levulinic acid is generally greater than 65%, preferably greater than 71%, very preferably greater than 79%.

During the synthesis, the solvent may undergo degradation into undesired byproducts. The degradation rate of the solvent Xₛₒₗ is defined as the ratio of the mass of solvent consumed to the mass of solvent initially used in the reaction medium, expressed in %. The mass of solvent consumed is defined as the difference between the measured mass of solvent and the mass of solvent initially used. The degradation rate of the solvent is generally less than 11%, preferably less than 8%, very preferably less than 5%.

At the end of the synthesis by hydration of furfuryl alcohol, a reaction effluent is obtained containing levulinic acid, water, homogeneous acid catalyst (preferably hydrochloric acid HCl), solvent (preferably 1,4-dioxane and/or 1,2-dimethoxyethane), and possibly traces of unconverted furfuryl alcohol, and also the unavoidable humins formed which are high-molecular-weight products resulting from condensation reactions, in particular by condensation of furfuryl alcohol with itself. The humins are soluble in the reaction medium.

### Separation Step (Optional)

In order to obtain levulinic acid with a high yield and high purity, the reaction effluent may be subjected to one or more separation steps. The levulinic acid can be separated from the reaction effluent by any method known to a person skilled in the art.

The separation and purification methods commonly used for separating levulinic acid from the reaction medium comprise solvent extraction, vacuum distillation, crystallization, ion exchange, membrane separation, etc. Such separation methods are described for example in WO2012/065115, WO2012/162028, WO2015/007602 or also CN107867996.

There are also separation processes based solely on thermal separation steps, such as distillation or evaporation. Document WO2018/235012 for example describes a separation and purification method involving two distillation steps.

Advantageously, the reaction effluent resulting from the synthesis according to the invention is subjected to at least one separation step.

Advantageously, the reaction effluent resulting from the synthesis according to the invention is subjected to at least one thermal separation step.

Advantageously, the reaction effluent resulting from the synthesis according to the invention is subjected to at least one step of thermal separation in the presence of a flux having a boiling point greater than that of the levulinic acid.

The act of using a flux having a boiling point greater than that of the levulinic acid during a thermal separation step makes it possible to separate the levulinic acid from the humins formed in particular during the synthesis of levulinic acid. The use of a flux makes it possible to significantly improve the recovery rate (yield) of levulinic acid compared to conditions in which the flux is not used.

Moreover, the use of a flux also makes it possible to control the viscosity of the humins, in particular by reducing their viscosity. Indeed, in the absence of a flux, the humins are often recovered as a solid at ambient temperature. The presence of a flux makes it possible to recover a heavy fraction containing the humins in liquid and viscous form at ambient temperature, thus facilitating the discharge thereof in the separation unit and therefore limiting fouling of the equipment.

### Preliminary Thermal Separation Step (Optional)

The reaction effluent may be subjected to a preliminary thermal separation step aimed at separating off the compounds having a boiling point lower than that of the levulinic acid.

The preliminary thermal separation step makes it possible to separate off a light fraction comprising the water, the homogeneous acid catalyst (in particular the hydrochloric acid) and the solvent. These compounds are preferably then condensed and recycled into the synthesis unit, which makes it possible firstly to limit the emissions and the environmental impact of the synthesis and secondly to limit the consumption of resources and ultimately the production cost of the levulinic acid. The act of using an ether-and/or acetal-based solvent having a limited degradation rate during the synthesis thus makes it possible to increase the possible recycling rate thereof.

The preliminary thermal separation step can be carried out according to any method known to a person skilled in the art. It can, for example, be carried out by distillation and/or by evaporation.

According to a variant, and when it is carried out by distillation, use can be made of a plate distillation column. The number of theoretical plates is generally between 1 and 50, preferably between 1 and 10.

The column-bottom distillation temperature is advantageously between 25 and 200° C., preferably between 50 and 180° C., and very preferably between 100 and 160° C.

The column-top distillation pressure is advantageously between 0.0001 and 0.2 MPa (between 1 mbara and 2 bara), preferably between 0.001 and 0.1 MPa (between 10 mbara and 1 bara), and very preferably between 0.004 and 0.05 MPa (between 40 mbara and 500 mbara).

According to another variant, and when the preliminary thermal separation step is carried out by distillation, use can also be made of a packed distillation column operating within the same temperature and pressure ranges.

According to another variant, and when the preliminary thermal separation step is carried out by evaporation, use can be made of one or more evaporators in series or in parallel; the evaporator(s) can be chosen for example from natural or forced circulation evaporators, falling or climbing film evaporators, agitated thin film evaporators, plate evaporators or multiple-effect evaporators. Preferably, at least two evaporators are used in series. Falling film or climbing film evaporators are known devices in which the heating and the conversion of the liquid to vapor are carried out within a plurality of tubes, themselves heated by a fluid (for example low-pressure steam), inside which the liquid flows in the form of a film along the inner wall of the tubes. The heat applied through the walls of each tube causes the light fraction of the liquid mixture to evaporate. In the case of a falling film evaporator, the film of liquid flows downwards, by virtue of the action of the force of gravity, whereas in the case of a rising film evaporator, the liquid film is pushed upwards by the vapor generated from the boiling. In this way, the liquid is heated rapidly, with high-temperature residence times that are quite reduced compared to distillation, and consequently a lower risk of degradation of the organic products present in the liquid itself.

The evaporator(s) operate within the same temperature and pressure ranges as described for the distillation.

The preliminary thermal separation step aims in particular to separate the compounds having a boiling point lower than that of the levulinic acid from the reaction effluent, making it possible to obtain a composition comprising levulinic acid and humins which is freed of the light compounds and which is, preferably at least in part, continuously or batchwise, introduced into another step of separation in the presence of a flux having a boiling point greater than that of the levulinic acid.

The preliminary thermal separation step can be carried out in the absence or in the presence of a flux as defined below, and preferably it is carried out in the absence of a flux.

The preliminary thermal separation step is generally carried out such that the content of water in the composition comprising levulinic acid and humins which is sent into the downstream thermal separation step is less than 1% by weight relative to the total weight of the composition, preferably less than 0.9% by weight and particularly preferably less than 0.8% by weight.

### Levulinic Acid Thermal Separation Step (Optional)

The reaction effluent comprising levulinic acid and humins, which has possibly been freed of the compounds having a boiling point lower than that of the levulinic acid by a preliminary thermal separation step, may then be subjected to an additional thermal separation step.

Preferably, this additional thermal separation step can be carried out in the presence of a flux having a boiling point greater than that of the levulinic acid, so as to obtain a light fraction containing the levulinic acid and a heavy fraction containing the humins and said flux.

Preferably, the reaction effluent no longer contains compounds having a boiling point lower than that of the levulinic acid during this step.

The flux should have a boiling point greater than that of the levulinic acid, which is 245° C., in order to be able to carry out the separation. The flux generally has a boiling point of greater than 250° C., preferably of greater than 280° C., and particularly preferably of greater than 300° C.

The flux should also be stable and should not degrade at a temperature of between 20 and 200° C., preferably between 150 and 200° C. Specifically, the flux should not degrade into compounds that might react with the levulinic acid or into lighter compounds that might be separated off with the light fraction containing the levulinic acid. The flux may contain polar or protic functions.

The flux may be of petroleum origin and/or of vegetable origin and/or based on polymers. The flux may also be a mixture of at least two of these components.

When the flux is of petroleum origin, it can be chosen from any petroleum cut having a boiling point greater than that of the levulinic acid, in particular from a vacuum gas oil (VGO) (which typically has a boiling range of from 360° C. to 620° C.), a settling oil or a recycle oil (which typically has a boiling range of from 360° C. to 620° C.), for example a fluidized-bed catalytic cracking effluent such as a heavy cycle oil (HCO), an unconverted oil originating from a hydrocracker which typically has a boiling range of from 360° C. to 620° C. (UCO), vacuum residues (which typically have a boiling range of greater than or equal to 524° C.), deasphalted oils, resins, or a mixture thereof.

When the flux is of vegetable origin, it may be chosen from a vegetable and/or animal oil or also from fatty acid methyl esters (FAMEs) which may be produced either by esterification of fatty acids derived from vegetable and/or animal oil or by direct transesterification of vegetable and/or animal oil, or a mixture thereof. Mention may for example be made of olive oils or avocado oil. This nonlimiting list also includes all oils obtained by genetic modification or hybridization. Spent oils, such as frying oils, and also all spent oils and fats from the catering industries, may also be used. With regard to animal fats, mention may be made, without being limiting, of tallow. The expressions “animal fat” and “animal oil” are used without distinction in the present description, the only difference between a fat and an oil being the state of the fatty substance at ambient temperature: liquid for an oil and solid for a fat.

These vegetable and/or animal oils may be crude or totally or partially refined. Typically, the distinction between a crude or a refined vegetable oil refers to the method by which it was extracted, mainly with pressing for a crude oil (typically a single pressing under cold conditions without additives) and generally using a solvent for a refined oil.

When the flux is based on polymers, it may be chosen from polyethers of the type of polyethylene glycol having an average molar mass of greater than or equal to 600 g/mol, and in particular PEG-600, PEG-800 or PEG 1000, or also PEG 6000 or PEG 8000, alone or as a mixture.

The flux is advantageously mixed with said reaction effluent before and/or during the performance of the thermal separation step. The mixing can be carried out before the separation unit or in the separation unit.

The mixing can be carried out by any means known to a person skilled in the art, for example a stirrer, recirculation of the liquid with a pump or by passing the mixture through a static mixer.

The amount of flux introduced into said mixture is such that the content by mass of flux in said mixture is between 0.5% and 85% by weight, preferably between 1% and 70% by weight, and with preference between 1.5% and 50% by weight, relative to the total weight of the mixture.

The mixing is advantageously carried out at a temperature of between 25 and 200° C., preferably of between 50 and 195° C., and very preferably of between 110 and 190° C.

The mixing time is advantageously between 0.1 and 600 minutes, preferably between 1 and 60 minutes.

In general, the flux makes it possible to reduce the viscosity of the humins contained in the heavy fraction. Indeed, in the absence of a flux, the heavy fraction containing the humins is often recovered as a solid at ambient temperature. The presence of a flux makes it possible to recover the heavy fraction in liquid and viscous form at ambient temperature, thus facilitating the discharge thereof in the separation unit and therefore limiting fouling of the equipment.

The flux also makes it possible to control the thermal separation of the levulinic acid by limiting the separation temperature and thus the formation of undesired secondary products of levulinic acid, such as angelica lactone. The presence of the flux thus makes it possible to significantly improve the recovery rate of levulinic acid via the conservation of the levulinic acid by limiting the separation temperature and by controlling the viscosity of the residue containing the humins, in particular by reducing its viscosity.

The step of thermal separation of the levulinic acid performed in the presence or absence of the flux can be carried out according to any method known to a person skilled in the art. It can, for example, be carried out by distillation and/or by evaporation.

According to a variant, and when it is carried out by distillation, use can be made of a plate distillation column. The number of theoretical plates is generally between 1 and 50, preferably between 1 and 20.

The column-bottom distillation temperature is advantageously between 80 and 200° C., preferably between 100 and 195° C., and very preferably between 110 and 190° C.

The column-top distillation pressure is advantageously between 0.0001 and 0.1 MPa (between 1 mbara and 1 bara), preferably between 0.001 and 0.08 MPa (between 10 mbara and 800 mbara), and very preferably between 0.002 and 0.05 MPa (between 20 mbara and 500 mbara).

According to another variant, and when the step of thermal separation of the levulinic acid in the presence or absence of the flux is carried out by distillation, use can also be made of a packed distillation column operating within the same temperature and pressure ranges.

According to another variant, and when the step of thermal separation in the presence of the flux is carried out by evaporation, use can be made of one or more evaporators in series or in parallel; the evaporator(s) can be chosen for example from natural or forced circulation evaporators, falling or climbing film evaporators, agitated thin film evaporators, plate evaporators or multiple-effect evaporators.

The evaporator(s) operate within the same temperature and pressure ranges as described for the preliminary distillation.

Advantageously, when the step of thermal separation in the presence of the flux is carried out by evaporation, use can be made of a thin film evaporator.

In contrast to a falling or rising film evaporator, a thin film evaporator comprises a single tube inside of which the liquid to be treated flows along the inner wall of the tube itself. The film of liquid is distributed uniformly over the wall by virtue of the action of a blade rotor inserted within the tube which, when it is set in rotation, in addition to distributing the liquid over the wall, creates a turbulent flow within the film itself, which considerably improves the heat exchange. This type of evaporator makes it possible to rapidly separate the most volatile part from the least volatile part by virtue of the agitation of the liquid in the form of a film under controlled conditions. The evaporator preferably operates at reduced pressure in order to lower the temperature of separation of the vapor phase from the liquid phase. The heating of the wall of the tube is carried out for example by external coils within which circulates a heating fluid, for example steam.

This type of equipment makes it possible to very significantly limit the residence time of the products/residues compared to a distillation column or falling or rising film evaporators. Specifically, an increase in the viscosity of the residue as a function of the time at high temperature (150-200° C.) is observed, which contributes to significant fouling of the separation equipment. The use of a thin film evaporator makes it possible to limit the residence time of said composition comprising levulinic acid and humins and therefore to significantly limit the increase in viscosity and hence to improve the operability of this separation step.

The recovery rate of levulinic acid, corresponding to the mass of levulinic acid vaporized in relation to the mass of levulinic acid contained in the composition used in the step of separation in the presence of a flux, is generally between 80% and 99%, and preferably between 82% and 99%.

The obtained purity of levulinic acid is between 90.0% and 99.0% by weight, preferably between 90.1% and 98.0% by weight, very preferably between 90.2% and 97.9% by weight. If necessary, and in order to increase the purity, the levulinic acid can be subjected to one or more purification steps, such as a crystallization or esterification step.

The light fraction comprising the levulinic acid can then be cooled and condensed. The cooling and the condensation of this fraction can then optionally be integrated into a production of low-pressure steam by heat exchange, thus enabling a saving of energy. The low-pressure steam can be used as heating in the evaporator(s).

The heavy fraction containing the humins and the flux is liquid and therefore easy to discharge from the separation unit. It can be sent to an external waste treatment. It can also be burned to produce thermal energy, for example for the separation unit(s).

According to a preferred variant, the reaction effluent resulting from the synthesis according to the invention and comprising levulinic acid and humins and compounds having a boiling point lower than that of the levulinic acid is subjected to a separation process comprising the following steps:
said effluent is subjected to a preliminary thermal separation step so as to separate off the compounds having a boiling point lower than that of the levulinic acid,
said effluent comprising levulinic acid and humins is then subjected to a step of thermal separation in the presence of a flux having a boiling point greater than that of the levulinic acid, so as to obtain a light fraction containing the levulinic acid and a heavy fraction containing the humins and said flux.

### LIST OF THE FIGURES

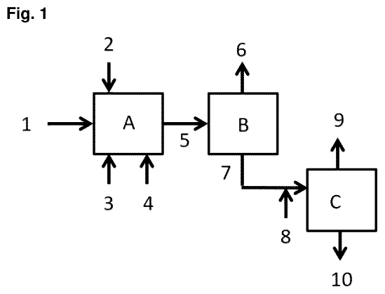

The information regarding the elements referenced in FIG. 1 enables a better understanding of the invention, without said invention being limited to the particular embodiments illustrated in FIG. 1. The various embodiments presented can be used alone or in combination with one another, without limitation of combination.

FIG. 1 represents the diagram of a preferred embodiment of the process of the present invention, comprising:
the synthesis of levulinic acid by hydration of furfuryl alcohol in the presence of a homogeneous acid catalyst in a continuously or batchwise operating reactor A in which furfuryl alcohol 1, water 2, hydrochloric acid 3 and the solvent 1,4-dioxane 4 are introduced and this mixture is heated in order to synthesize the levulinic acid.

The reaction effluent 5 is sent continuously or batchwise into a preliminary thermal separation section B which can be implemented by distillation or evaporation. The preliminary thermal separation step B makes it possible to separate a light fraction 6 containing the hydrochloric acid, the solvent and the unconverted water, which can be at least partly recycled into the reactor A (recycle not shown), and a heavy fraction (residue) 7 comprising the levulinic acid and the humins freed of light compounds. This composition is mixed with a flux 8 and then this mixture is sent into a thermal separation section C which can be implemented by distillation or evaporation and which makes it possible to obtain a light fraction containing the levulinic acid 9 and a heavy fraction 10 containing the humins and the flux.

### EXAMPLES

The following examples are carried out according to the protocol below.

A flask with a capacity of 500 mL and equipped with a condenser is charged with 172 g of a solvent, 20 g of water (1.11 mol) and 20 g of an aqueous 37% by weight solution of hydrochloric acid (0.7 mol of water and 0.21 mol of HCl). The mixture is brought to a temperature of 80° C. under magnetic stirring. 132 g of furfuryl alcohol (1.35 mol) are then poured into the flask over a duration of addition of 6 h by means of a peristaltic pump. At the end of the addition, the mixture is left to react at the reaction pressure and temperature for a maturation phase of 15 minutes, before being cooled. The final mixture is taken for analysis and the concentrations by mass of solvent and of levulinic acid present are quantified by ¹H NMR spectroscopy calibrated by addition of a known amount of acetonitrile. The theoretical final concentrations by mass of levulinic acid and solvent are 45.4% and 50.0%, respectively.

Example 1 (not in accordance with the invention) uses methyl ethyl ketone (MEK) as solvent. The measured yield of levulinic acid is 82%, and the degradation rate of the solvent is 12%.

Example 2 (in accordance with the invention) uses 1,4-dioxane as solvent. The measured yield of levulinic acid is 87%, and the degradation rate of the solvent is 1%.

Example 3 (in accordance with the invention) uses 1,2-dimethoxyethane as solvent. The measured yield of levulinic acid is 82%, and the degradation rate of the solvent is 4%.

Thus, the examples presented here confirm that the use of a solvent of ether or acetal type makes it possible to obtain equivalent or even better yields compared to those obtained in ketones such as MEK, while at the same time significantly limiting the degradation of the solvent by maintaining the rate below a value of 5%.

TABLE 1

| | | Final LA | Final solvent | | |
|---|---|---|---|---|---|
| | | concen- | concen- | | |
| | | tration | tration | Yield_{LA} | Xₛₒₗ |
| Example | Solvent | (% wt) | (% wt) | (%) | (%) |
| 1 (not in | MEK | 37.4 | 44.1 | 82 | 12 |
| accordance | | | | | |
| with the | | | | | |
| invention) | | | | | |
| 2 (in | 1,4- | 39.5 | 49.8 | 87 | 1 |
| accordance | dioxane | | | | |
| with the | | | | | |
| invention) | | | | | |
| 3 (in | 1,2- | 37.4 | 48.0 | 82 | 4 |
| accordance | dimethoxy- | | | | |
| with the | ethane | | | | |
| invention) | | | | | |

1. A process for synthesizing levulinic acid by hydration of furfuryl alcohol at a temperature of between 25 and 140° C. in the presence of a homogeneous acid catalyst and of an ether- and/or acetal-based solvent.

2. The process as claimed in claim 1, wherein the ether- and/or acetal-based solvent is chosen from the compounds corresponding to one or the other of the structures I and II, taken alone or as a mixture: 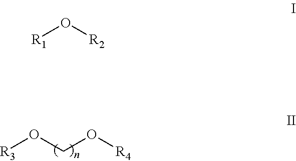
in which R₁, R₂, R₃ and R₄ are independently chosen from:
linear or branched aliphatic groups of 1 to 6 carbon atoms, optionally substituted by alkoxy groups,
cyclic or polycyclic aliphatic groups of 5 to 12 carbon atoms, optionally substituted by alkoxy or alkyl groups,
linear or branched olefinic groups of 1 to 6 carbon atoms, optionally substituted by alkoxy groups,
1 aromatic or polyaromatic groups of 6 to 12 carbon atoms,
R₁ and R₂ may be bonded together by covalent bonds so as to form a ring,
R₃ and R₄ may be bonded together by covalent bonds so as to form a ring,
n is an integer between 1 and 6.

3. The process as claimed in claim 1, wherein the solvent is chosen from diethyl ether, diisopropyl ether, diisobutyl ether, dibutyl ether, diphenyl ether, 2-methoxy-2-methylpropane, 2-methoxy-2-methylbutane, 2,5-dihydrofuran, tetrahydrofuran, 2-methyltetrahydrofuran, 3-methyltetrahydrofuran, 2,3-dihydropyran, tetrahydropyran, 1,3-dioxolane, 1,3-dioxane, 1,4-dioxane, 1,2-dimethoxyethane, benzofuran, 2,2-dimethoxypropane, 2,2-di(2-ethylhexyloxy) propane, 2-methoxytetrahydrofuran and di(2-methoxyethyl) ether, taken alone or as a mixture.

4. The process as claimed in claim 1, wherein the homogeneous acid catalyst is chosen from a homogeneous, organic or inorganic Brønsted acid.

5. The process as claimed in claim 1, wherein the homogeneous acid catalyst is hydrochloric acid.

6. The process as claimed in claim 1, wherein water is present in an amount such that the water/furfuryl alcohol molar ratio is between 0.9 and 10.0 mol/mol.

7. The process as claimed in claim 1, wherein the solvent is present in an amount such that the solvent/furfuryl alcohol molar ratio is between 0.1 and 5 mol/mol.

8. The process as claimed in claim 1, wherein the homogeneous acid catalyst is present in an amount such that the acid/furfuryl alcohol molar ratio is between 0.01 and 1.0 mol/mol.

9. The process as claimed in claim 1, which is carried out at a temperature of between 60 and 110° C.

10. The process as claimed in one of the preceding claims, claim 1, which is carried out at a pressure of between 0.01 MPa and 1 MPa.

11. The process as claimed in claim 1, wherein the reaction effluent resulting from the synthesis is subjected to at least one separation step.

12. The process as claimed in claim 1, wherein the reaction effluent resulting from the synthesis is subjected to at least one thermal separation step.

13. The process as claimed in claim 1, wherein the reaction effluent resulting from the synthesis is subjected to at least one step of thermal separation in the presence of a flux having a boiling point greater than that of the levulinic acid.

14. The process as claimed in claim 13, wherein the flux has a boiling range of between 250 and 620° C. and is of petroleum origin and/or of vegetable origin and/or based on polymers or a mixture thereof.

15. The process as claimed in claim 13, wherein the flux is chosen from a petroleum cut chosen from a vacuum gas oil, a heavy oil obtained from a fluidized-bed catalytic cracking, a settling oil, an unconverted oil originating from a hydrocracker, or a polyethylene glycol having an average molar mass of greater than or equal to 600 g/mol.
